(19) United States
(12) Patent Application Publication
(10) Pub. No.: US 2013/0102811 A1
(43) Pub. Date: Apr. 25, 2013

(54) PROCESS FOR THE PREPARATION OF LACOSAMIDE

(71) Applicants: Maheshbhai Mehulkumar Patel, Baroda (IN); Vishal Diliprao Mohite, Baroda (IN); Sudhakar Khambampati, Baroda (IN); Rao Trinadha Chitturi, Baroda (IN); Rajamannar Thennati, Baroda (IN)

(72) Inventors: Maheshbhai Mehulkumar Patel, Baroda (IN); Vishal Diliprao Mohite, Baroda (IN); Sudhakar Khambampati, Baroda (IN); Rao Trinadha Chitturi, Baroda (IN); Rajamannar Thennati, Baroda (IN)

(73) Assignee: SUN PHARMACEUTICAL INDUSTRIES LTD, Mumbai (IN)

(21) Appl. No.: 13/807,849

(22) PCT Filed: Jul. 1, 2011
(86) PCT No.: PCT/IN2011/000437

§ 371 Date: Dec. 31, 2012

(30) Foreign Application Priority Data
Jul. 2, 2010 (IN) 1929/MUM/2010

(51) Int. Cl. C07C 231/02

(52) U.S. Cl. CPC C07C 231/02
USPC 564/158

(57) ABSTRACT

The present invention relates to a novel and improved process for the preparation of lacosamide, wherein the process is a sequential one-pot process.

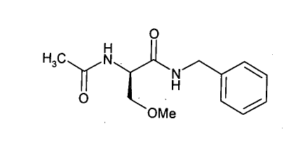

### RELATED APPLICATION

This application claims the benefit of Indian Patent Application No. 1929/MUM/2010 filed on Jul. 02, 2010 which is hereby incorporated by reference.

### FIELD OF INVENTION

The present invention relates to a novel and improved process for the preparation of lacosamide.

### BACKGROUND OF THE INVENTION

Lacosamide is a chiral molecule, which is chemically (R)-2-acetamido-N-benzyl-3-methoxypropionamide having the following structure:

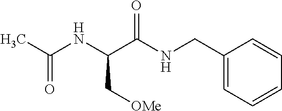 [SMILES: COC[C@@H](NC(C)=O)C(=O)NCc1ccccc1]

It is an anticonvulsant drug useful in the treatment of central nervous system disorders such as epilepsy. The drug is also useful in the treatment of pain, particularly neuropathic pain such as diabetic neuropathic pain. It was approved by

USFDA in October 2008 as an adjunctive therapy for partial-onset seizures and is marketed by UCB under the trade name VIMPAT®.

Lacosamide was specifically disclosed in U.S. Pat. No. 38,551 (referred to as '551 patent hereinafter). The '551 patent discloses three methods for the preparation of lacosamide as depicted in Schemes 1, 2 and 3 below, and provides detailed process steps using these three schemes.

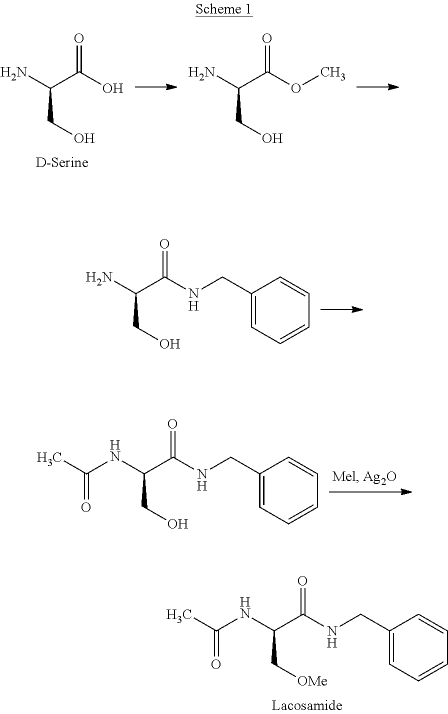

Scheme 1 involves the formation of benzylamide of D-serine, followed by acetylation and then methylation using methyl iodide-silver oxide to give lacosamide. Scheme 2 of the '551 patent involves N-acetylation of D-serine to yield N-acetyl derivative, followed by benzylamide formation and subsequent methylation to give the final product, lacosamide.

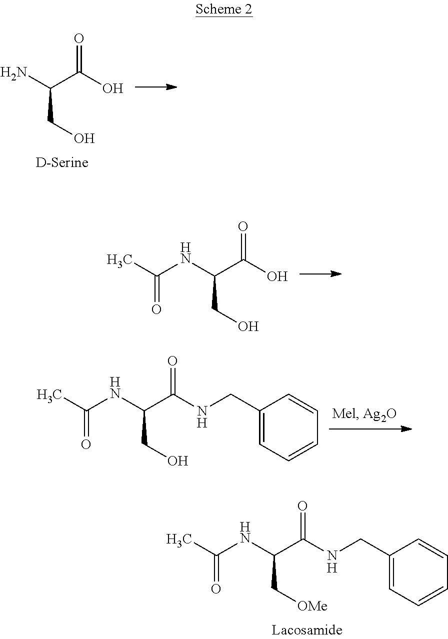

Scheme 3 involves protection of the amino group by carbobenzyloxy group, methylation of the N-protected intermediate resulting in a product which is both O-methylated at the —OH and esterified at the —COOH group. Hydrolysis of the ester yields the desired O-methylated product, which is subjected to benzylamide formation, deprotection and final N-acetylation to give lacosamide. The reaction sequence of Scheme 3 is as depicted below:

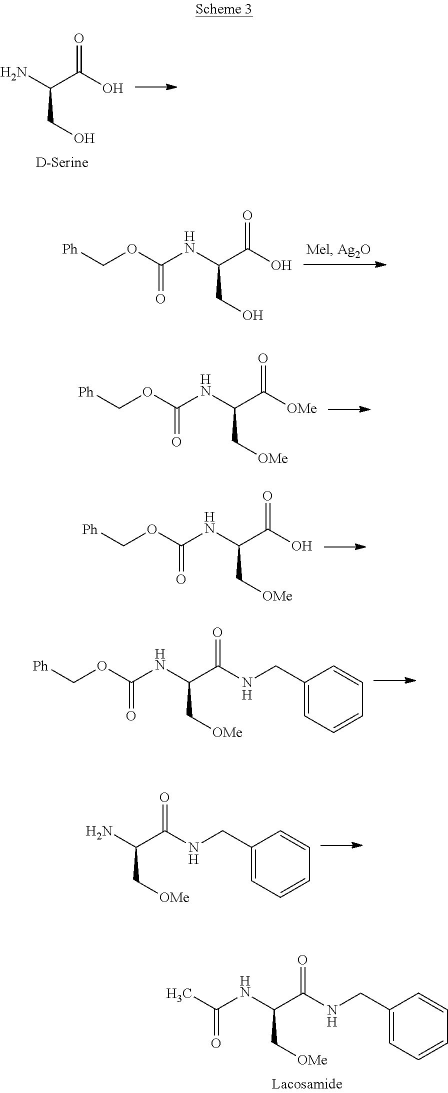

These processes of the prior art suffer from several disadvantages, particularly the use of expensive reagents like methyl iodide and silver oxide for methylation, besides requiring long reaction times. For example, one of the steps requires as long as 4 days for completion and generates several impurities, which necessitates use of chromatography for purification. Further, the process involves isolation of intermediates at each step of the synthesis which further leads to increase in turnaround time. All these factors are non-conducive for industrial scale manufacture.

The above mentioned processes are also exemplified in U.S. Pat. No. 6,048,899, which is a continuation-in-part of the '551 patent. As indicated in example 1 of the '899 patent, the process results in partial racemization of the product. Further, methylation of the hydroxy group also results in esterification of the free carboxylic acid group, which is then de-esterified in an additional step to yield the desired methylated product. Furthermore, the process involves additional purification steps to be performed i.e. salt formation with (R)-mandelic acid for removal of the enantiomer impurity and purification of final product by preparative chromatography. The process results in low yield of lacosamide and chiral purity of not more than 95%.

US2008/0027137 (referred to as '137 hereinafter) also identifies the problem of racemization and esterification, as mentioned above, in the prior art processes. The '137 patent application discloses a process for preparing lacosamide comprising O-methylation of N-Boc-D-serine to obtain O-methyl-N-Boc-D-serine which is then sequentially subjected to benzylamide formation, deprotection reaction to remove the Boc protecting group and acetylation to yield lacosamide, as depicted in Scheme 4 below:

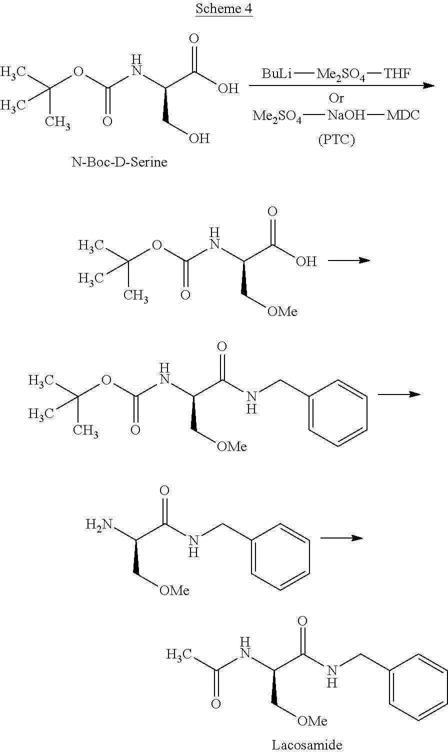

The process described in the '137 application attempts to overcome the formation of ester occurring during the O-methylation step in the prior art processes by providing a “one-step” reaction i.e. a process wherein O-methylation is carried out in a single step without formation of the esterified product thereby avoiding an additional de-esterification reaction. The process goes through O-methylated N-Boc-D-serine intermediate which however has partial solubility in water, therefore requiring repeated extractions with dichloromethane for its isolation. Furthermore, the process teaches use of oganolithium compounds as bases for the O-methylation reaction. However, these oraganolithioum compounds are hazardous and expensive, and their use especially for manufacture on large scale is not desirable.

The abstract of CN101591300 discloses yet another different route for the preparation of lacosamide comprising O-methylation of N-protected D-serine using methyl iodide and silver oxide, acetylation and then benzylamide formation to prepare lacosamide as a one pot process. As mentioned for the '551 patent above, the process once again involves methylation of N-protected D-serine using expensive reagents, prior to benzylamide formation. Besides, there are chances of esterification during the methylation step. Thus, there is a need for an improved, cost-effective process for preparation of lacosamide with high chiral purity on commercial scale.

The present invention involves a novel and improved process for preparation of lacosamide. The process does not yield the unwanted ester product and is simpler to carry out when compared to the prior art processes. The process according to the present invention, besides requiring less reaction times, is cost effective in that it does not use expensive reagents like silver oxide or hazardous reagents like organolithium compounds unlike the prior art processes. Also, the process does not require elaborate purifications or use of chromatography, and thus is more suitable for industrial scale manufacture. Furthermore, the product is obtained in good yields with high purity, including high chiral purity.

### SUMMARY OF THE INVENTION

In one embodiment, the present invention relates to a process for preparation of (R)-2-acetamido-N-benzyl-3-methoxypropionamide (lacosamide) which comprises the steps of:
a) condensing N-Boc-D-serine with benzylamine to obtain the compound of formula I

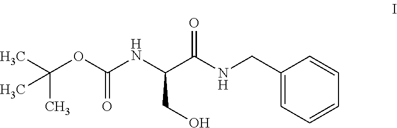 [SMILES: CC(C)(C)OC(=O)N[C@H](CO)C(=O)NCc1ccccc1]

b) methylating the compound of formula I to prepare a compound of formula II

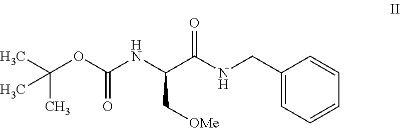 [SMILES: COC[C@@H](NC(=O)OC(C)(C)C)C(=O)NCc1ccccc1]

c) hydrolyzing the compound of formula II to the amino compound of formula III, and

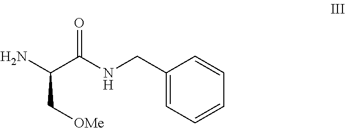 [SMILES: COC[C@@H](N)C(=O)NCc1ccccc1]

d) acylating the compound of formula III to obtain (R)-2-acetamido-N-benzyl-3-methoxypropionamide (lacosamide)

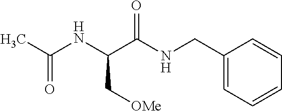 [SMILES: COC[C@@H](NC(C)=O)C(=O)NCc1ccccc1]

wherein the process is a sequential one-pot process.

In a preferred embodiment the reactions from step a) to d) are carried out in a single organic solvent.

In another embodiment, the present invention relates to a process of preparation of N-Boc-D-serine comprising reacting in water an in situ generated alkali metal salt of D-serine with t-butyloxycarbonic anhydride.

In yet another embodiment, the present invention relates to a process which yields lacosamide in high chiral purity of ≧99.0%, preferably >99.9%.

### DETAILED DESCRIPTION OF THE INVENTION

In one embodiment, the present invention relates to a process of preparing lacosamide comprising the steps a) to d) above, wherein the process is performed sequentially in a single pot

Step a) of the process involves condensing N-Boc-D-serine with benzylamine to obtain the compound of formula I

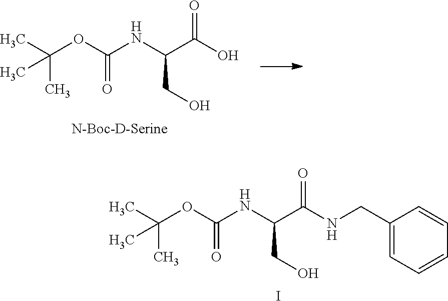

The process is performed in the presence of a base, a coupling agent in an organic solvent, optionally in the presence of catalytic amount of 1-hydroxybenzotriazole. The base that may be used in this reaction may be a tertiary organic base selected from triethylamine, diisopropylamine, pyridine, N-alkylmorpholines etc. In a preferred embodiment, the base is N-methylmorpholine. The coupling agent or the carboxyl group activator may be selected from the group consisting of (benzotriazole-1-yloxy)tris (dimethylamino)phosphonium hexafluorophosphate (BOP), isobutyl chloroformate, N,N′-dicyclohexylcarbodiimide (DCC) and N-(3-dimethylaminopropyl)-N-ethylcarbodiimide (EDC). In a preferred embodiment, the coupling agent is isobutyl chloroformate.

The solvent for the reaction may be a water immiscible aprotic solvent such as toluene, ethyl acetate or dichloromethane, preferably the solvent is dichloromethane.

Step b) of the process involves O-methylating the compound of formula I to prepare a compound of formula II

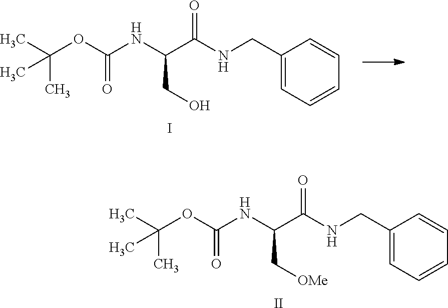

The O-methylation reaction is carried out by adding a methylating agent to the biphasic system comprising solution of compound of formula I in a water immiscible organic solvent as mentioned above, and an aqueous solution of an inorganic base; and catalytic amount of a phase transfer catalyst.

The methylating agent may be selected from dimethyl sulfate, methyl triflate, and trimethyl phosphate, Preferred methylating agent for the reaction is dimethyl sulfate.

The phase transfer catalyst used in the reaction may be a quarternized amine salt, or a phosphonium salt. The quarternized amine salt may be selected from tetraalkylammonium salts such as sulfate, chloride or bromide; benzyltrialkylammonium halides, cetyltrialkylammonium halides; Tweens (polyoxyethylene sorbitan esters) such as Tween®20, Tween®40, Tween®60, Tween®80, Tween®85 etc. The phosphonium salt is preferably selected from triphenylmethyl triphenylphosphonium chloride, benzyltriphenylphosphonium chloride, butyltriphenylphosphonium bromide, ethyltriphenylphosphonium bromide, ethyltriphenylphosphonium iodide, methyltriphenylphosphonium bromide, methyltriphenylphosphonium iodide, tetraphenylphosphonium bromide. Preferably, the phase transfer catalyst is a quarternized ammonium salt, for e.g. tetrabutylammonium bromide. The amount of the phase transfer catalyst that may be used is about 0.01 to 0.10 mole equivalent, preferably 0.02 to 0.05 mole equivalent with reference to compound of formula I.

The inorganic base is present in the aqueous phase and may be selected from an alkali metal hydroxide, carbonate or bicarbonate. The base is preferably an alkali metal hydroxide, most preferred being sodium hydroxide.

Step c) of the process involves hydrolyzing the compound of formula II to the amino compound of formula III.

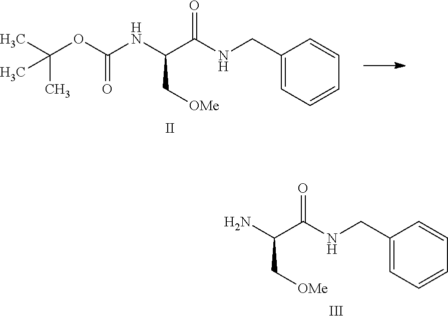

The hydrolysis of the compound leads to deprotection of the amino group. This reaction may be performed in presence of an organic or an inorganic acid. The organic acid may be selected from a carboxylic or a sulfonic acid such as trifluoroacetic acid, trifluoromethanesulfonic acid, methanesulfonic acid, formic acid, p-toluenesulfonic acid etc. Preferably the organic acid may be methanesulphonic acid. The inorganic acid may be selected from a mineral acid such as hydrochloric acid, hydrobromic acid, sulfuric acid, phosphoric acid etc. Preferably the inorganic acid is hydrochloric acid.

Step d) involves acetylation of the free amino compound of formula III to form lacosamide. N-Acetylation may be performed using acetic anhydride in presence of an inorganic base in the water immiscible aprotic solvent as mentioned in step a). The inorganic base that may be used according to the present process may be selected from a carbonate or bicarbonate of an alkali metal. Preferably the inorganic base is potassium carbonate.

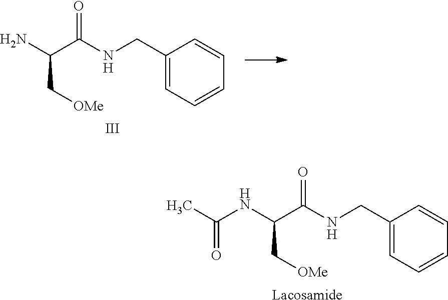

In another embodiment, the present invention relates to process of preparing N-Boc-D-serine, which is used as a starting material in step a) by reacting a solution of alkali metal salt of D-serine with t-butyloxycarbonic anhydride, optionally in presence of a phase transfer catalyst. Preferably, the reaction is performed in the absence of any organic solvent.

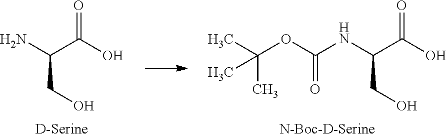

The Phase transfer catalyst that may be used for the reaction may be a quarternized amine salt and a phosphonium salt. The preferred catalyst and the quantity that may be used is as described in the preparation of compound of formula II from compound of formula I, vide supra. Upon completion of the reaction, the product N-Boc-D-serine is extracted into a water immiscible organic solvent as mentioned in step a), and the product containing extract may be used directly for step a) of the process without requiring to isolate the product.

In a preferred embodiment of the present invention, all the reactions from steps a) to step d) are carried out in the same single organic solvent. Preferably the solvent is a water immiscible aprotic solvent such as toluene, ethyl acetate or dichloromethane, most preferably the solvent is dichloromethane.

In yet another embodiment, the process provides optional method for enhancement of chiral purity of lacosamide comprising leaching the product with water.

In yet another embodiment of the present invention, lacosamide is obtained in high chiral purity ≧99.0%, preferably >99.9%. This chiral purity is achieved without the need of a separate process for resolution of the desired enantiomer.

The examples that follow do not limit the scope of the present invention and are included as illustrations

### EXAMPLES

### Example 1: (R)-N-Boc-D-Serine

To a stirred solution of sodium hydroxide 114 g (2.85 mole) in water (375 ml) was added (R)-serine 250 g (2.37 mole). To the resulting clear solution of sodium salt of (R)-serine were added t-butyloxycarbonic anhydride (Boc-anhydride) 571 g (2.61 mole) and a catalytic amount tetrabutylammonium bromide 12.0 g (0.036 mole). The mixture was stirred at ambient temperature for 16 hours. The resulting suspension was acidified to pH 3.5-4.0 with dilute HCl (3N) and the product was extracted into dichloromethane to obtain a solution of (R)-N-Boc-serine.

### Example 2: (R)-N-Benzyl-2-N-Boc-Amino-3-Hydroxypropionamide

To the above solution of (R)-N-Boc-serine in dichloromethane was added N-methylmorpholine 264.7 g (2.61 mole), cooled to −20° C., and added isobutyl chloroformate 357.4 g (2.617 mole) followed by benzylamine 382 g (3.56 mole). The mixture was then stirred at ambient temperature for 1 hour, washed with dilute HCl (1 N) to obtain a solution of (R)-N-Boc-serine benzamide in dichloromethane.

### Example 3: (R)-N-Benzyl-2-N-Boc-Amino-3-Methoxypropionamide

To the above solution of (R)-N-benzyl-2-N-Boc-amino-3-hydroxypropionamide in dichloromethane was added 50% w/w aqueous solution of sodium hydroxide 250 ml (4.75 mole) and tetrabutylammonium bromide 12.0 g (0.036 mole). Cooled to 5° C., added dimethyl sulfate 389.5 g (3.09 mole) and stirred at ambient temperature for 2 hrs. The aqueous layer was separated, and the organic layer was washed with water to obtain a solution of (R)-N-benzyl-2-N-Boc-amino-3-methoxypropionamide in dichoromethane.

### Example 4: (R)-2-Amino-N-Benzyl-3-Methoxypropionamide

To the above solution of (R)-N-benzyl-2-N-Boc-amino-3-methoxypropionamide in dichoromethane was added conc. hydrochloric acid and stirred at ambient temperature for 1 hour. Water was then added to the mixture, stirred and separated the product containing aqueous layer. Basified the aqueous layer to pH 10-12 and extracted with dichloromethane to obtain a solution of (R)-2-amino-N-benzyl-3-methoxypropionamide in dichloromethane.

### Example 5: Lacosamide

To the above solution of (R)-2-Amino-N-benzyl-3-methoxypropionamide was added potassium carbonate 164.0 g (1.18 mole) and acetic anhydride 237 g (2.32 mole) at 0-5° C. The reaction mixture was then stirred at ambient temperature for 1 hour and washed with water. The organic layer was concentrated, the residue stripped once with ethyl acetate and then crystallized from ethyl acetate to obtain lacosamide with HPLC purity>99%, chiral purity using chiral HPLC was 99%.

Repeat crystallization from ethyl acetate provides lacosamide with chiral purity>99.5%.

### Example 6: Chiral Enrichment of Lacosamide

A sample of lacosamide, 1.1 g, containing 1.3% of (S)-isomer was stirred as a thick suspension in DM water for 1 hour, filtered and dried to obtain lacosamide with chiral purity 99.96%.

### Example 7: (R)-2-Amino-N-Benzyl-3-Methoxypropionamide from (R)-N-Benzyl-2-N-Boc-Amino-3-Methoxypropionamide by Using Methanesulfonic Acid

To the solution of (R)-N-benzyl-2-N-Boc-amino-3-methoxypropionamide 8.66 g (0.028 mole) in dichloromethane was added of 30% v/v aqueous methanesulfonic acid 14.5 ml and stirred at ambient temperature for 18 hours. Water was added to the mixture, stirred and separated the aqueous layer. Basified the aqueous layer to pH 10-12 and extracted with dichloromethane to obtain a solution of (R)-2-amino-N-benzyl-3-methoxypropionamide in dichloromethane.

The solution of (R)-2-amino-N-benzyl-3-methoxypropionamide in dichloromethane was converted to lacosamide in the same manner as described in example 5.

1. A process for preparation of (R)-2-acetamido-N-benzyl-3-methoxypropionamide (Lacosamide) wherein the process is sequential a one-pot process, 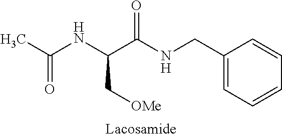
comprising,
a) condensing N-Boc-D-serine with benzylamine to obtain the compound of formula I, 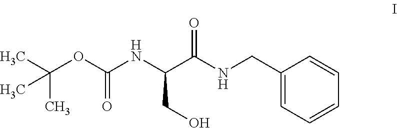 [SMILES: CC(C)(C)OC(=O)N[C@H](CO)C(=O)NCc1ccccc1]
b) methylating the compound of formula Ito prepare a compound of formula II, 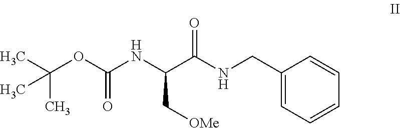 [SMILES: COC[C@@H](NC(=O)OC(C)(C)C)C(=O)NCc1ccccc1]
c) hydrolyzing the compound of formula II to the amino compound of formula III, and 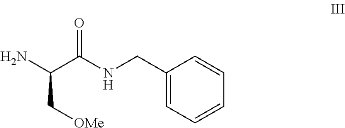 [SMILES: COC[C@@H](N)C(=O)NCc1ccccc1]
d) acylating the compound of formula III to obtain (R)-2-acetamido-N-benzyl-3-methoxypropionamide (lacosamide).

2. The process as claimed in claim 1, wherein all the reactions of step a) to d) are carried out in dicholormethane.

3. The process as claimed in claim 1, wherein the condensation of N-Boc-D-serine with benzylamine in step a) is performed using a coupling agent, optionally in the presence of catalytic 1-hydroxybenzitriazole

4. The process as claimed in claim 3, wherein the coupling agent is selected from the group consisting of (benzotriazole- 1-yloxy)tris(dimethylamino)phosphonium hexafluorophosphate (BOP), is obutyl chloroformate, N,N′-dicyclohexylcarbodiimide (DCC) and N-(3-dimethylaminopropyl)-N-ethylcarbodiimide(EDC).

5. The process as claimed in claim 1, wherein methylation of compound of formula I in step b) is carried out using a methylating agent in a biphasic system in presence of a phase transfer catalyst.

6. The process as claimed in claim 5, wherein the methylating agent is selected from the group consisting of dimethyl sulfate, methyl triflate, and trimethyl phosphate.

7. The process as claimed in claim 6, wherein the methylating agent is dimethyl sulfate.

8. The process as claimed in claim 5, wherein the aqueous phase of the biphasic system contains an inorganic base.

9. The process as claimed in claim 8, wherein the inorganic base is selected from an alkali metal hydroxide, carbonate and bicarbonate.

10. The process as claimed in claim 5, wherein the phase transfer catalyst is selected from a quarternized amine salt or a phosphonium salt.

11. The process as claimed in claim 10, wherein quarternized amine salt is selected from the group consisting of sulfate, chloride or bromide salts of tetraalkylammonium; benzyltrialkylammonium halides; cetyltrialkylammonium halides and Tweens (polyoxyethylene sorbitan esters) such as Tween®20, Tween®40, Tween®60, Tween®80 and Tween®85.

12. The process as claimed in claim 10, wherein the phosphonium salt is selected from the group consisting of triphenylmethyl triphenylphosphonium chloride, benzyltriphenylphosphonium chloride, butyltriphenylphosphonium bromide, ethyltriphenylphosphonium bromide, ethyltriphenylphosphonium iodide, methyltriphenylphosphonium bromide, methyltriphenylphosphonium iodide and tetraphenylphosphonium bromide.

13. The process as claimed in claim 10, wherein the phase transfer catalyst is tetrabutylammonium bromide.

14. The process as claimed in claim 5, wherein the phase transfer catalyst is used is about 0.01 to 0.10 mole equivalent with reference to compound of formula I.

15. The process as claimed in claim 14, wherein the phase transfer catalyst 0.02 to 0.05 mole equivalent.

16. The process as claimed in claim 1, wherein the hydrolysis of compound of formula II in step c) is carried out using an organic acid or a mineral acid.

17. The process as claimed in claim 16, wherein the organic acid is selected from a carboxylic or a sulfonic acid consisting of trifluoroacetic acid, trifluoromethanesulfonic acid, methanesulfonic acid, formic acid or p-toluenesulfonic acid.

18. The process as claimed in claim 17, wherein the mineral acid is selected from hydrochloric acid, hydrobromic acid, sulfuric acid and phosphoric acid.

19. The process as claimed in claim 1, wherein acylation of compound of formula III in step d) is carried out using acetic anhydride in the presence of an inorganic base.

20. The process as claimed in claim 19, wherein the inorganic base is selected from carbonate or bicarbonates of an alkali metal.

21. The process as claimed in claim 20, wherein the inorganic base is potassium carbonate.

22. The process as claimed in claim 1, wherein N-Boc-D-serine used in step a) is prepared by reacting an aqueous solution of an alkali metal salt of D-serine with t-butyloxycarbonic anhydride in the absence of a solvent, optionally in the presence of a phase transfer catalyst

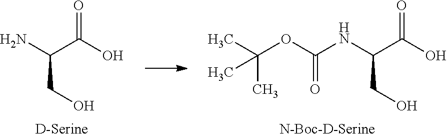

23. The process as claimed in claim 1, wherein the process further involves optional enhancement of chiral purity of lacosamide by leaching with water.

24. The process as claimed in claim 1, wherein (R)-2-acetamido-N-benzyl-3-methoxypropionamide (Lacosamide) is obtained in a chiral purity of ≧99.0%.

25. The process as claimed in claim 1, wherein (R)-2-acetamido-N-benzyl-3-methoxypropionamide (Lacosamide) is obtained in a chiral purity of >99.9%.
